(19) United States
(12) Patent Application Publication
(10) Pub. No.: US 2019/0008773 A1
(43) Pub. Date: Jan. 10, 2019

(54) MAGNETIC FIELD SENSITIVE NANO COMPLEX AND METHOD FOR MANUFACTURING THE SAME

(71) Applicant: GWANGJU INSTITUTE OF SCIENCE AND TECHNOLOGY, Gwangju (KR)

(72) Inventors: Jae-Young LEE, Gwangju (KR); Guru Karthikeyan Thirunavukkarasu, Gwangju (KR); Hwang-Jae LEE, Gwangju (KR)

(21) Appl. No.: 16/031,076

(22) Filed: Jul. 10, 2018

(30) Foreign Application Priority Data
Jul. 10, 2017 (KR) 10-2017-0086923

(51) Int. Cl. A61K 9/14; A61K 31/65; A61P 31/00

(52) U.S. Cl. CPC A61K 9/146; A61P 31/00; A61K 31/65; A61K 9/143

(57) ABSTRACT

Disclosed herein is a magnetic field-sensitive nanocomposite and a method of preparing the same. The magnetic field-sensitive nanocomposite includes a biodegradable polyester-based polymer particle, a magnetic nanoparticle dispersed in the biodegradable polyester-based polymer particle, and a drug loaded in the biodegradable polyester-based polymer particle.

### CROSS-REFERENCE TO RELATED APPLICATION

This application claims priority under 35 U.S.C. to Korean Application No. 10-2017-0086923, filed on Jul. 10, 2017, whose entire disclosure is herein incorporated by reference.

### BACKGROUND

### 1. Field of the Invention

The present disclosure relates to a magnetic field-sensitive nanocomposite, and more particularly, to a magnetic field-sensitive nanocomposite for anticancer treatment and a method of preparing the same.

### 2. Description of Related Art

Various methods have been studied for diagnosis and treatment of cancer. A drug-based treatment has been commonly used. Research has been actively conducted to improve problems such as selective delivery, side effects and efficiency of a drug.

In order to enhance the effectiveness of chemotherapy, an effective drug delivery system should be used to effectively deliver a drug to a site where treatment is required. In addition, a stimulation-sensitive release system is required to enhance the efficacy of the delivered drug.

In particular, a system for releasing a drug depending on an external stimulus advantageously makes it possible to perform a flexible and active treatment based on a treatment progress. Furthermore, an imaging technique that shows the treatment progress of a cancer cell may also serve to enhance the efficacy of a cancer treatment.

In the meantime, there has been used a system configured to apply light from the outside using a material having high absorbance such as a gold nanoparticle, graphene oxide and the like, and release a loaded drug in response to the applied light.

However, the material such as a gold nanoparticle or graphene oxide is an untested material. Such a material may be unsuitable for direct use in clinical practice. This material itself cannot be imaged, and thus it is required to perform an additional reforming step and add a compound for imaging.

In particular, light used to induce drug release may transmit only a few millimeters (mm) even when the light is a near-infrared ray having the highest transmittance.

In addition, a conventional method of loading a drug is performed mainly through physical adsorption. As a result, the drug is released due to spontaneous diffusion. The chemotherapy has a limitation in that it is difficult to adjust an amount of the drug being released.

The background art is disclosed in Korean Patent Publication No. 10-1351331 (granted on Jan. 7, 2014), and the aforementioned document discloses a synthesis method of magnetic nanoparticles for a targetable drug delivery system and a drug delivery vector using the same.

### SUMMARY OF THE INVENTION

An aspect of the present disclosure provides a magnetic field-sensitive nanocomposite for anticancer treatment.

Another aspect of the present disclosure provides a method of preparing the magnetic field-sensitive nanocomposite for anticancer treatment.

The magnetic field-sensitive nanocomposite according to an aspect of the present disclosure may include a biodegradable polyester-based polymer article, a magnetic nanoparticle dispersed in the biodegradable polyester-based polymer particle, and a drug loaded in the biodegradable polyester-based polymer particle.

The biodegradable polyester-based polymer particle may include at least one of poly (L-lactic acid) (PLA), polyglycolic acid (PGA) and polylactic-co-glycolic acid) PLGA.

The biodegradable polyester-based polymer particle may have a glass transition temperature (Tg) of 50° C. or less.

The magnetic field-sensitive nanocomposite may include 1 to 80 parts by weight of the magnetic nanoparticle with respect to 100 parts by weight of the polymer particle.

The magnetic nanoparticle may have an average diameter of 1 to 50 nm,

The magnetic nanoparticle may include a superparamagnetic iron oxide nanoparticle.

The drug may include at least one of a diagnostic drug, a therapeutic drug, and a drug for a reaction reagent.

The biodegradable polyester-based polymer particle may have an average diameter of 900 nm or less.

The method of preparing a magnetic field-sensitive nanocomposite according to another aspect of the present disclosure may include (a) mixing an organic solvent, a magnetic nanoparticle and a biodegradable polyester-based polymer, (b) adding distilled water in which a drug is dispersed to a resultant product of the step (a) and stirring the resultant product with the distilled water to form a first emulsion, (c) adding distilled water in which an emulsifier is dissolved to the first emulsion and stirring the first emulsion with the distilled water to form a second emulsion, and (d) drying the second emulsion to prepare a magnetic field-sensitive nanocomposite. The magnetic field-sensitive nanocomposite may include the biodegradable polyester-based polymer particle, a magnetic nanoparticle dispersed in the polymer particle, and a drug loaded in the polymer particle. The magnetic nanoparticle may generate heat when an alternating magnetic field is applied to the magnetic field-sensitive nanocomposite, and the generated heat may cause the biodegradable polyester-based polymer particle to undergo a decomposition such that the drug is released.

In the step (a), 1 to 80 parts by weight of the magnetic nanoparticle may be mixed with respect to 100 parts by weight of the biodegradable polyester-based polymer.

In each of the steps (b) and (c), the stirring may be performed using ultrasonic waves.

The biodegradable polyester-based polymer may include at least one of PLA, PGA, and PLGA.

The biodegradable polyester-based polymer may have a glass transition temperature (Tg) of 50° C. or less.

The magnetic nanoparticle may have an average diameter of 1 to 50 nm.

The magnetic nanoparticle may include a superparamagnetic iron oxide nanoparticle.

The drug may include at least one of a diagnostic drug, a therapeutic drug, and a drug for a reaction reagent.

A system for anticancer treatment according to still another aspect of the present disclosure may include the magnetic field-sensitive nanocomposite according to claim 1, and the magnetic nanoparticle may generate heat when an alternating magnetic field is applied to the magnetic field-sensitive nanocomposite, and the generated heat may cause the biodegradable polyester-based polymer particle to undergo a phase-transition or decomposition such that the drug is released.

The magnetic field-sensitive nanocomposite according to the present disclosure may use a magnetic nanoparticle and a biodegradable polyester-based polymer particle which is sensitive to a rise in temperature resulting from an exothermic reaction of the magnetic nanoparticle as a drug delivery system. Accordingly, the magnetic field-sensitive nanocomposite may receive an external alternating magnetic field to induce selective heat generation and drug delivery simultaneously. In particular, the magnetic nanoparticle and the biodegradable polyester-based polymer may be decomposed in vivo, and thus harmless to a human body. Therefore, the magnetic field-sensitive nanocomposite according to the present disclosure may be used as a material for effective cancer treatment.

When an external alternating magnetic field is applied to the magnetic field-sensitive nanocomposite, the magnetic nanoparticle may generate heat, and the generated heat may be primarily used to treat a cancer cell. The polymer may be phase-transited at a temperature higher than the glass transition temperature by the generated heat, whereby it is possible to release the drug and perform a secondary chemotherapy to treat a cancer cell.

Further, the magnetic nanoparticle according to the present disclosure may perform a magnetic resonance (MR) imaging simultaneously, and thus penetrate deeply into the body. Therefore, the magnetic field-sensitive nanocomposite according to the present disclosure is effective for cancer treatment.

### BRIEF DESCRIPTION OF THE DRAWINGS

FIG. 1 is a schematic diagram showing a process in which hyperthermia treatment of cancer is performed and a process in which a loaded drug is released by an alternating magnetic field applied to a magnetic field-sensitive nanocomposite according to the present disclosure.

FIG. 2 is a schematic diagram showing a method of preparing a magnetic field-sensitive nanocomposite according to the present disclosure.

FIG. 3A and FIG. 3B are photographs of a magnetic field-sensitive nanocomposite according to the present disclosure, and FIG. 3C is a graph showing results of analyzing diameters of an iron oxide nanoparticle and a magnetic field-sensitive nanocomposite through a dynamic laser scattering (DLS) method.

FIG. 4A is a graph showing results of measuring peaks of components included in a magnetic field-sensitive nanocomposite according to the present disclosure using a fourier transform infrared spectroscopy. FIG. 4B is a graph showing a superparamagnetic property of a magnetic field-sensitive nanocomposite measured through a vibrating sample magnetometer (VSM).

FIG. 5A is a graph showing a phase-transition of a magnetic field-sensitive nanocomposite according to the present disclosure using a differential scanning calorimetry (DSC). FIG. 5B is a graph showing a temperature rise of an iron oxide nanoparticle resulting from an external alternating magnetic field.

FIG. 6A is a graph showing drug release being induced in line with a temperature rise of a magnetic field-sensitive nanocomposite according to the present disclosure, and FIG. 6B and FIG. 6C are TEM photographs showing a state of a magnetic field-sensitive nanocomposite after a drug is released.

FIG. 7 is a graph showing that a temperature of a magnetic field-sensitive nanocomposite according to the present disclosure rises and a drug is acceleratedly released every time an alternating magnetic field is periodically applied to the magnetic field-sensitive nanocomposite.

FIG. 8A is a graph showing cytotoxicity in a physiological environment of a magnetic field-sensitive nanocomposite according to the present disclosure. FIG. 8B is a graph showing viability of a cancer cell when an alternating magnetic field (AMF) is applied.

### DETAILED DESCRIPTION OF EXEMPLARY EMBODIMENTS

Advantages and features of the present disclosure and methods for achieving them will become apparent from the descriptions of embodiments herein below with reference to the accompanying drawings. However, the present disclosure is not limited to the embodiments disclosed herein but may be implemented in various different forms. The embodiments are provided to make the description of the present disclosure thorough and to fully convey the scope of the present disclosure to those skilled in the art. It is to be noted that the scope of the present disclosure is defined only by the claims. In the drawings, same reference numerals designate same or like elements.

Hereinafter, a magnetic field-sensitive nanocomposite according to preferred embodiments of the present disclosure and a method of preparing the same will be described in detail with reference to the accompanying drawings.

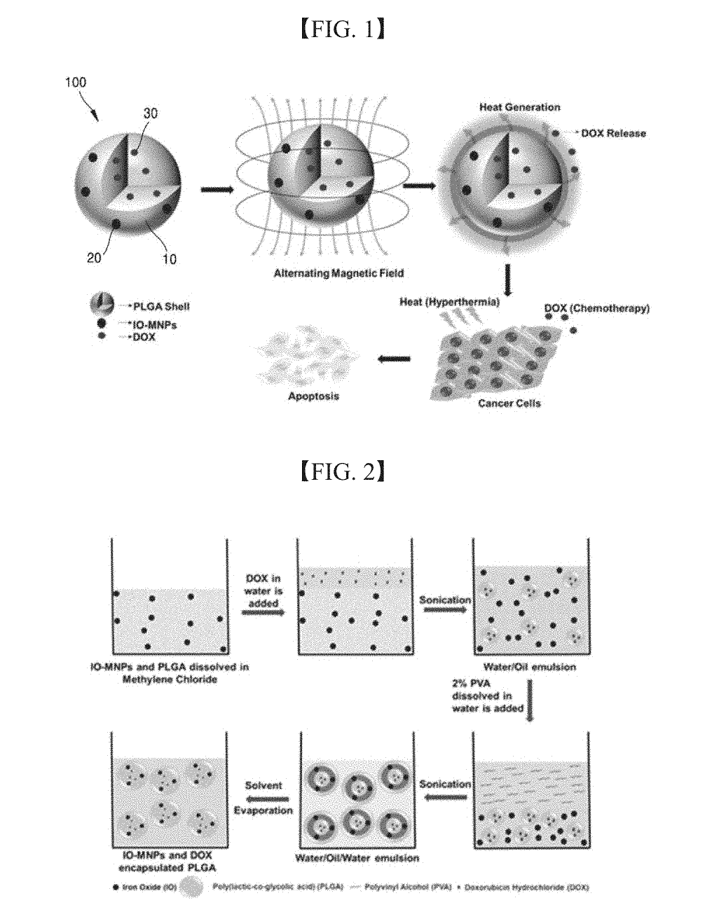

FIG. 1 is a schematic diagram showing a process in which hyperthermia treatment of cancer is performed and a process in which a loaded drug is released by an alternating magnetic field applied to a magnetic field-sensitive nanocomposite according to the present disclosure.

Referring to FIG. 1, a magnetic field-sensitive nanocomposite according to the present disclosure may include a biodegradable polyester-based polymer particle 10, a magnetic nanoparticle 20 dispersed in the biodegradable polyester-based polymer particle 10 and a drug 30 loaded in the biodegradable polyester-based polymer particle 10.

As shown in FIG. 1, the magnetic nanoparticle 20 may generate heat when an alternating magnetic field is applied to the magnetic field-sensitive nanocomposite 100. And, the generated heat may cause the biodegradable polyester-based polymer particle 10 to undergo a phase-transition or decomposition such that the drug 30 is released.

Biodegradable Polyester-Based Polymer Particle 10

The biodegradable polyester-based polymer may be a thermosensitive polymer. The biodegradable polyester-based polymer may exhibit thermal sensitivity accompanied by changes in a structure and physical properties occurring at a glass transition temperature (TG) when a temperature thereof rises.

The biodegradable polyester-based polymer may preferably have a glass transition temperature (Tg) of 50° C. or less. more preferably 35 to 45° C. When an alternating magnetic field is applied from the outside, the polymer may be phase-transited within this temperature range, and the polymer particle may be decomposed to release a drug loaded therein.

For example, the biodegradable polyester-based polymer particle may include at least one of poly(L-lactic acid) (PLA), polyglycolic acid (PGA), and polylactic-co-glycolic acid) (PLGA). Preferably, the PLGA may be used as the biodegradable polyester-based polymer particle.

The biodegradable polyester-based polymer particle may have a structure in which magnetic nanoparticle and a drug to be described later may be dispersed therein. The biodegradable polyester-based polymer particle may be formed in a spherical shape.

Considering that the magnetic field-sensitive nanocomposite according to the present disclosure is nano-sized, the biodegradable polyester-based polymer particle may preferably have an average diameter of 900 nm or less, more preferably 100 to 300 nm.

When the average diameter of the polymer particle exceeds 900 nm, a cancer cell is less likely to endocytose the polymer particle, resulting in a relatively low cancer treatment effect. Further, it is difficult to disperse the polymer particle, resulting in difficulty in obtaining homogeneous and reproducible results. In addition, when the average diameter of the magnetic field-sensitive nanocomposite is extended, an in vivo residence time may be reduced, thereby reducing a pharmacological effect of the composite.

Magnetic Nanoparticle 20

The magnetic nanoparticle 20 may be dispersed in the biodegradable polyester-based polymer particle 10, and generate heat by alternating magnetic field applied from the outside.

That is, the magnetic nanoparticle 20 may have a characteristic of generating heat under the alternating magnetic field, and thus may be used for hyperthermia treatment of cancer. In addition, the magnetic nanoparticle 20 may have characteristics of being excellent in sensitivity, non-toxic and rapidly discharged from the body as a magnetic resonance (MR) contrast agent.

The magnetic nanoparticle 20 may preferably include a superparamagnetic iron oxide nanoparticle. For example, the magnetic nanoparticle 20 may include at least one of Fe304, CoFe2O4, NiFe2O4, CuFe2O4, ZnFe2O4, MgFe3O4 and MnFe3O4.

In general, paramagneticity may be hardly magnetized even when a magnetic field is applied from the outside.

On the other hand, super paramagneticity may have a large magnetic moment. Thus, when a magnetic field is applied from the outside, the super paramagneticity may have strong magnetism such as ferromagneticity.

The magnetic nanoparticle 20 may preferably have an average diameter of 1 to 50 nm. The magnetic nanoparticle 20 having an average diameter within the above-described range may exhibit excellent superparamagneticity. That is, when the superparamagnetic iron oxide nanoparticle has an average diameter of 1 to 50 nm, it may exhibit excellent superparamagneticity. In addition, the superparamagnetic iron oxide nanoparticle may exhibit excellent dispersibility in the polymer particle 10 without being agglomerated.

The magnetic field-sensitive nanocomposite 100 may include 1 to 80 parts by weight of the magnetic nanoparticle 20 with respect to 100 parts by weight of the polymer particle 10. When a content of the magnetic nanoparticle 20 is less than 1 part by weight, it may be difficult to perform hyperthermia treatment because heat generated in the magnetic field-sensitive nanocomposite 100 is not sufficient. On the contrary, when the content of the magnetic nanoparticle 20 exceeds 80 parts by weight, the density of the magnetic field-sensitive nanocomposite 100 may increase due to an excessive content of the magnetic nanoparticle 20. Further, it may be difficult to adjust drug release due to sudden heat generation of the magnetic nanoparticle 20.

Also, a surface of the magnetic nanoparticle 20 may be coated with a hydrophobic material such as oleic acid, but is not limited thereto.

Drug 30

The drug (30) may be a drug loaded in the biodegradable polyester-based polymer particle 10. The drug 30 may include at least one of a diagnostic drug, a therapeutic drug, and a drug for a reaction reagent.

For example, the drug 30 may be a biguanides-based compound including metformin, chlorpropamide, glibenclamide (glyburide), a sulfonylureas-based compound including gliclazide, glimepiride, glipizide, gliquidone, tolazamide and tolbutamide, an alpha-glucosidase inhibitor including acarbose, miglitol and voglibose, a meglitinides-based compound including nateglinide, repaglinide and mitiglinide, a dipeptidyl peptidase-4 (DPP-4) inhibitor including alogliptin, saxagliptin, sitagliptin and vildagliptin, insulin, or the like.

In addition, the drug 30 may include an anticancer agent such as epirubicin, docetaxel, gemcitabine, paclitaxel, cisplatin, carboplatin, taxol, procarbazine, cyclophosphamide, dactinomycin, diunorubicin, etoposide, tamoxifen, doxorubicin, mitomycin, bleomycin, plicomycin, transplatinum, vinblastine or the like.

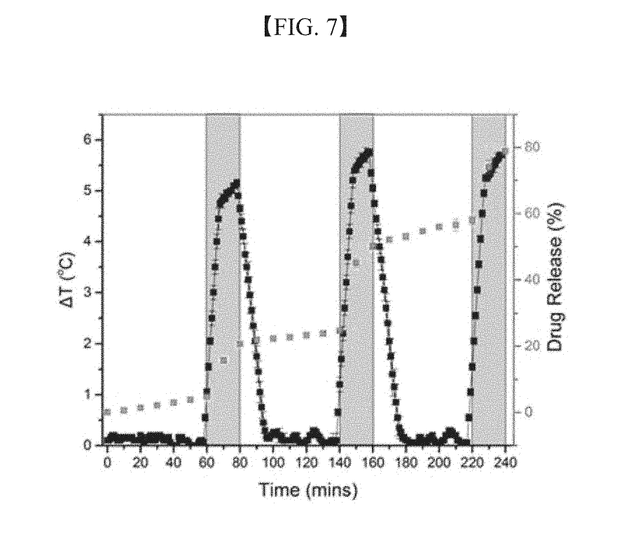

As described above, an alternating magnetic field may be periodically applied to the magnetic field-sensitive nanocomposite 100 for about 15 to 25 minutes. At the time of applying the alternating magnetic field, the larger a change in temperature, the greater an amount of the drug released, which can be ascertained from the graph of FIG. 7.

The application of the alternating magnetic field may be performed as follows For example, an alternating magnetic field inducing device may be connected to a coil. An output adjusting terminal located in the alternating magnetic field inducing device may be adjusted to generate a magnetic field. Accordingly, an alternating magnetic field may be formed around the coil through the alternating magnetic field inducing device, and the alternating magnetic field may be applied to a human body.

The alternating magnetic field may be applied by generating an alternating voltage having a frequency of about 10 kHz to 200 MHz, but is not limited thereto.

FIG. 2 is a schematic diagram showing a method of preparing a magnetic field-sensitive nanocomposite according to the present disclosure.

Referring to FIG. 2, a method of preparing a magnetic field-sensitive nanocomposite may include (a) mixing an organic solvent, a magnetic nanoparticle and a biodegradable polyester-based polymer, (b) adding distilled water in which a drug is dispersed to a resultant product of the step (a), and stirring the resultant product with the distilled water to form a first emulsion, (c) adding distilled water in which an emulsifier is dissolved to the first emulsion and stirring the first emulsion with the distilled water to form a second emulsion, and (d) drying the second emulsion to prepare a magnetic field-sensitive nanocomposite.

The magnetic field-sensitive nanocomposite may include the biodegradable polyester-based polymer particle, a magnetic nanoparticle dispersed in the polymer particle, and a drug loaded in the polymer particle. The magnetic nanoparticle may generate heat when an alternating magnetic field is applied to the magnetic field-sensitive nanocomposite, and the generated heat may cause the biodegradable polyester-based polymer particle to undergo a phase-transition or decomposition such that the drug is released.

(a) Mixing an Organic Solvent, a Magnetic Nanoparticle and a Biodegradable Polyester-Based Polymer

As shown in FIG. 2, an organic solvent, a magnetic nanoparticle and a biodegradable polyester-based polymer may be mixed. Any volatile solvent may be used as the organic solvent. The volatile solvent may be a solvent that dissolves the biodegradable polyester-based polymer. Further, the volatile solvent may be a solvent that is easily removed through evaporation. The organic solvent may serve not only as a dissolving agent for dissolving the biodegradable polyester-based polymer but also as a dispersing agent.

The organic solvent may be, for example, at least one of methylene chloride, ethyl acetate, chloroform, acetone, dimethylsulfoxide, dimethylformamide, N-methylpyrrolidone, dioxane, tetrahydrofuran, ethyl acetate, methyl ethyl ketone and acetonitrile. The organic solvent may preferably be methylene chloride, ethyl acetate or chloroform.

As described above, 1 to 80 parts by weight of the magnetic nanoparticle may be mixed with respect to 100 parts by weight of the biodegradable polyester-based polymer.

The magnetic nanoparticle and the biodegradable polyester-based polymer may have the same configuration as described above.

(b) Adding Distilled Water in which a Drug is Dispersed to a Resultant Product of the Step a) and Stirring the Resultant Product with the Distilled Water to Form a First Emulsion

Subsequently, distilled water in which the drug is dispersed may be added to a resultant product of step (a), followed by stirring. The stirring may be performed using ultrasonic waves for about 10 minutes to 10 hours.

The ultrasonic waves each may have an output of about 20 to 2000W. The ultrasonic waves each may have a frequency of about 10 to 200 kHz. When the stirring is performed using the ultrasonic waves within the above-described ranges, there is no contamination caused by a medium. Also, since the biodegradable polyester polymer is pulverized using ultrasonic waves within the above-described ranges, it is possible to form a uniform particle size of the polymer when a second emulsion is formed.

Also, since the pulverized polymer, the magnetic nanoparticle, the organic solvent and the distilled water are stirred using ultrasonic waves, it is possible to form a first emulsion with a uniform distribution of the polymer, the magnetic nanoparticle, the organic solvent and the distilled water.

The first emulsion may be an emulsion in which the organic solvent, the distilled water, the biodegradable polyester-based polymer particle 10, the magnetic nanoparticle 20 and the drug 30 are uniformly dispersed. The first emulsion may have stability that a dispersed state is hardly fluctuated even after a lapse of time.

As described above, the drug 30 may include at least one of a diagnostic drug, a therapeutic drug, and a drug for a reaction reagent.

(c) Adding Distilled Water in which an Emulsifier is Dissolved to the First Emulsion and Stirring the First Emulsion with the Distilled Water to Form a Second Emulsion

Subsequently, a second emulsion may be formed from the first emulsion.

In order to stably complexity the biodegradable polyester-based polymer particle 10, the magnetic nanoparticle 20 and the drug 30 dispersed in the first emulsion, an emulsifier may be preferably added to the first emulsion.

The emulsifier may be a hydrophilic emulsifier dispersed in the distilled water. The emulsifier may include at least one of twin, triton, breeze, polyvinyl pyrrolidone, and polyvinyl alcohol, and may preferably include polyvinyl alcohol (PVA).

The emulsifier may be included in an amount of 10 to 30 parts by weight of with respect to 100 parts by weight of the biodegradable polyester-based polymer, but is not limited thereto.

As described above, the ultrasonic waves each may have an output of about 20 to 2000W. The ultrasonic waves each may have a frequency of about 10 to 200 kHz. Since the polymer, the magnetic nanoparticle, the organic solvent and the distilled water are stirred together with the emulsifier using the ultrasonic waves, it is possible to form a second emulsion with a uniform distribution of the polymer, the magnetic nanoparticle, the organic solvent, distilled water and the emulsifier. The second emulsion may have stability that a dispersed state is hardly fluctuated even after a lapse of time.

In the process of forming the second emulsion, the biodegradable polyester-based polymer may be granulated. The drug 30 may be loaded in the polymer particle 10.

Therefore, the magnetic nanoparticle 20 may uniformly dispersed in the biodegradable polyester-based polymer particle 10, and the drug 30 may be stably loaded by the stirring using the ultrasonic waves and the emulsifier.

(d) Drying the Second Emulsion to Prepare a Magnetic Field-Sensitive Nanocomposite.

Subsequently, the organic solvent included in the second emulsion may be removed to prepare a magnetic field-sensitive nanocomposite 100.

The biodegradable polyester-based polymer particle 10 may be included in the magnetic field-sensitive nanocomposite 100. Accordingly, the second emulsion may be preferably dried at a temperature lower than the glass transition temperature of the polymer particle 10. That is, it is preferable to perform lyophilization at a temperature of −20° C. or less for about 5 to 36 hours

A system for anticancer treatment according the present disclosure may include the magnetic field-sensitive nanocomposite. The magnetic nanoparticle may generate heat by applying an alternating magnetic field to the magnetic field-sensitive nanocomposite, and the generated heat may cause the biodegradable polyester-based polymer particle to undergo a decomposition such that the drug is released.

As described above, according to the present disclosure, it is possible to prepare a magnetic field-sensitive nanocomposite from the magnetic nanoparticle and the biodegradable polyester-based polymer particle using an emulsion synthesis method. The drug may be stably loaded in the biodegradable polyester-based polymer particle, whereby imaging of a cancer cell, hyperthermia treatment and chemotherapy may be performed simultaneously. In addition, it is advantageously possible to deliver a drug to a cancer cell deep within the body and perform hyperthermia treatment.

That is, an alternating magnetic field having a high permeability relative to the body may be applied to the magnetic field-sensitive nanocomposite so as to generate heat, whereby it is possible to perform hyperthermia treatment. The hyperthermia treatment is based on a principle that the biodegradable polyester-based polymer is phase-transited due to a temperature rise caused by the generated heat, resulting in the release of the drug loaded therein.

Therefore, the magnetic field-sensitive nanocomposite according to the present disclosure may be used for the hyperthermia treatment using an alternating magnetic field simultaneously with MR imaging. Also, the magnetic field-sensitive nanocomposite may induce drug release with excellent adjusting capability through the phase-transition of the biodegradable polyester-based polymer, and thus may be used for effective anticancer treatment.

In the following, specific embodiments of the magnetic field-sensitive nanocomposite will be described.

1. Preparation of a Magnetic Field-Sensitive Nanocomposite

Preparation Example of an Iron Oxide Nanoparticle

4.3 g of FeCl₂.4H₂O and 11.6 g of FeCl₃.6H₂O were added to 350 ml of distilled water and heated to 80° C. in a nitrogen gas atmosphere, and then mixed. Thereafter, 20 ml of NH₄OH was added thereto quickly, and mixed for 5 minutes. Then, 1 ml of oleic acid was added thereto and mixed for 25 minutes. After completion of a reaction, black precipitate was formed. Remaining oleic acid was removed therefrom by using distilled water, ethanol and acetone, and then dried in an oven to prepare iron oxide nanoparticles (IO-MNPs) coated with oleic acid.

### Embodiments

3.3 parts by weight of the prepared iron oxide nanoparticles (IO-MNPs) were dissolved in 20 ml of a methylene chloride solvent, and 100 parts by weight of PLGA was added thereto. 1 part by weight of doxorubicin (DOX) was dissolved in 4 ml of distilled water, mixed with the above-described solution, and subjected to sonication at a frequency of 200 kHz for 30 minutes to form a water/oil emulsion,

Subsequently, 120 ml of a 2% PVA solution was added thereto and subjected to the sonication at a frequency of 200 kHz for 5 minutes to form a water/oil/water emulsion, and the methylene chloride solvent was evaporated under stirring for 24 hours to prepare an IO/PLGA/DOX composite.

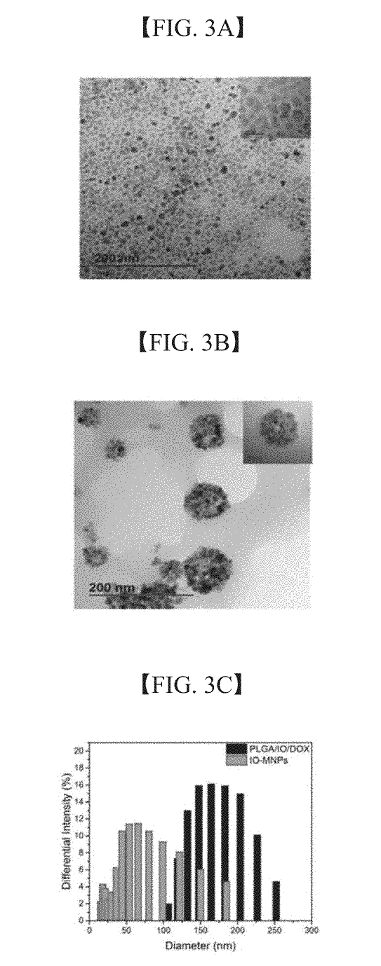

FIGS. 3A and 3B are photographs of a magnetic field-sensitive nanocomposite according to the present disclosure, and FIG. 3C is a graph showing results of analyzing diameters of an iron oxide nanoparticle and a magnetic field-sensitive nanocomposite through a dynamic laser scattering (DLS) method.

Referring to FIGS. 3A to 3C, the diameter and shape of the magnetic field-sensitive nanocomposite can be seen. From the graph of FIG. 3C, it can be ascertained that the magnetic field-sensitive nanocomposite has a diameter of about 150 to 200 nm.

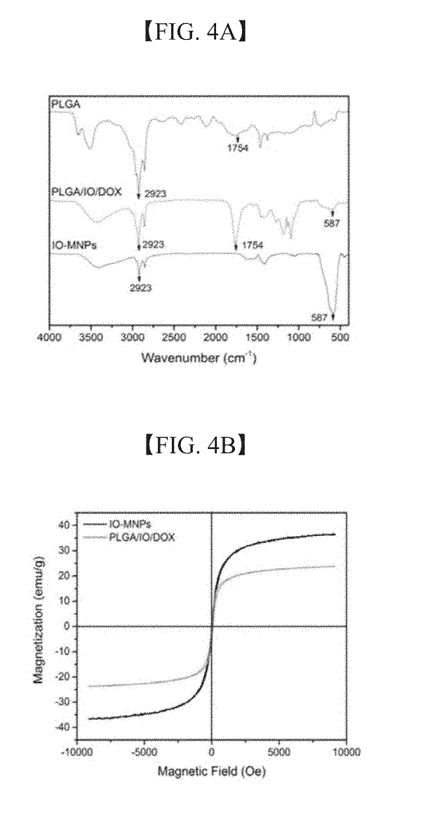

FIG. 4A is a graph showing results of measuring peaks of components included in a magnetic field-sensitive nanocomposite according to the present disclosure using a fourier transform infrared spectroscopy. FIG. 4B is a graph showing a superparamagnetic property of a magnetic field-sensitive nanocomposite measured through a vibrating sample magnetometer (VSM).

Referring to FIG. 4A, peaks inherent in PLGA and iron oxide were measured, from which, it can be ascertained that PLGA, an iron oxide nanoparticle, and DOX are included in the magnetic field-sensitive nanocomposite. Referring to FIG. 4B, it can be ascertained that the magnetic field-sensitive nanocomposite exhibits a superparamagnetic property and a degree thereof is about 20 emu/g.

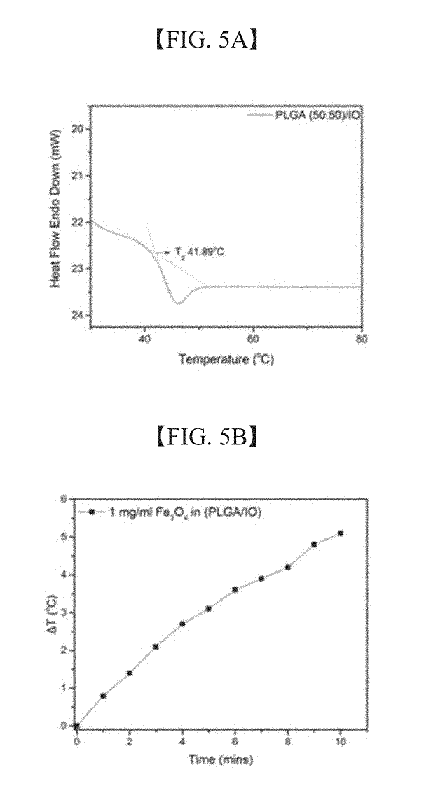

FIG. 5A is a graph showing a phase-transition of a magnetic field-sensitive nanocomposite according to the present disclosure using a differential scanning calorimetry (DSC). FIG. 5B is a graph showing a temperature rise of an iron oxide nanoparticle resulting from an external alternating magnetic field.

FIG. 5A illustrates that glass transition of PLGA occurs at around 42° C., from which, it can be anticipated that the drug release of the composite may be adjusted. Further, referring to 5B, it can be ascertained that the temperature of the iron oxide nanoparticles rises under an alternating magnetic field. It can be anticipated that the drug release may be adjusted thereby.

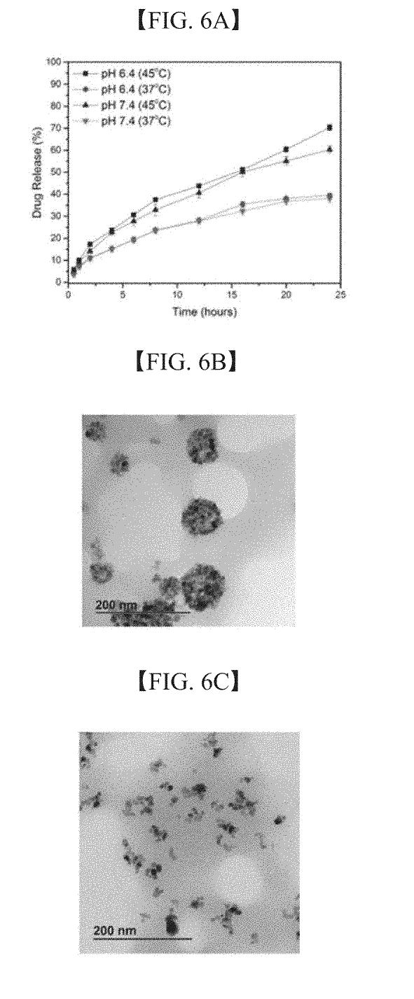

FIG. 6A is a graph showing drug release being induced in line with a temperature rise of a magnetic field-sensitive nanocomposite according to the present disclosure. FIGS. 6B and 6C are TEM photographs showing a state of a magnetic field-sensitive nanocomposite after a drug is released.

Referring to FIG. 6A, it can be ascertained that drug release is induced by a temperature rise of the magnetic field-sensitive nanocomposite whereas the drug release is not greatly influenced by pH from the result that a larger amount of doxorubicin is released at a temperature of 45° C. higher than a normal human body temperature of 37° C.

From a degraded from of a PLGA/IO/PLGA composite shown in the TEM photographs of FIGS. 6B and 6C, it can be ascertained that the PLGA was phase-transited at 45° C. equal to or higher than the glass transition temperature of the PLGA, and thereby the drug was released.

FIG. 7 is a graph showing that a temperature of a magnetic field-sensitive nanocomposite according to the present disclosure rises and a drug is acceleratedly released every time an alternating magnetic field is periodically applied to the magnetic field-sensitive nanocomposite. FIG. 7 shows a temperature change and an amount of a doxorubicin drug released by the temperature change when an alternating magnetic field is periodically applied to a sample including an IO/PLGA/DOX composite.

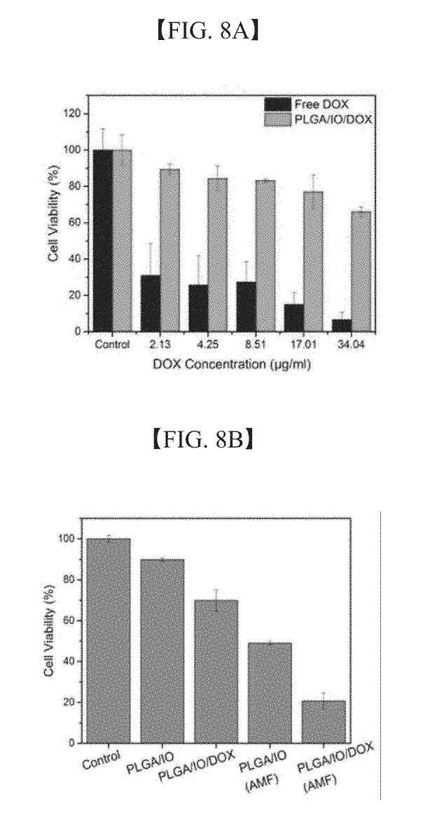

FIG. 8A is a graph showing cytotoxicity in a physiological environment of a magnetic field-sensitive nanocomposite according to the present disclosure. FIG. 8B is a graph showing viability of a cancer cell when an alternating magnetic field (AMF) is applied.

Referring to FIG. 8A, when DOX is used (free DOX) solely, non-specific cytotoxicity may be high. Thus, toxicity may be observed in other cells than a cancer cell.

Accordingly, a side effect of a drug may occur. On the other hand, when a drug is loaded in an IO/PLGA composite (PLGA/IO/DOX), the viability of a cell may be 60% or more. This result shows that the toxicity of the drug does not affect the surrounding cells in the process of delivering the drug and in the absence of stimulation of an alternating magnetic field.

In addition, the drug-loaded IO/PLGA composite makes it possible to combine the hyperthermia treatment with the chemotherapy, resulting in the highest efficiency of cancer cell treatment.

Referring to FIG. 8B, when the alternating magnetic field is applied to the drug-loaded IO/PLGA composite (PLGA/IO/DOX), it exhibits a superior effect of treating a cancer cell.

The combined treatment of the hyperthermia and the chemotherapy using the PLGA/IO/DOX composite may show a superior efficacy in comparison to the chemotherapy using a drug-loaded composite and the hyperthermia treatment using a drug-free IO/PLGA composite.

In FIG. 8B, when the alternating magnetic field (AMF) is applied to the PLGA/IO/DOX composite, the viability of a cancer cell may be 30% or less (the lowest), which shows a superior effect of treating a cancer cell.

Although the embodiments of the present disclosure have been described with reference to the accompanying drawings, the present disclosure is not limited to the embodiments, but may be modified into various forms. It will be understood by those skilled in the art that various changes can be made without departing from the scope of the present disclosure. Therefore, it should be understood that the embodiments described above are illustrative in all aspects and not restrictive.

### DESCRIPTION OF SYMBOLS

10: Biodegradable polyester-based polymer particle

20: Magnetic nanoparticle

30: Drug

100: Magnetic field-sensitive nanocomposite

What is claimed is:

1. A magnetic field-sensitive nanocomposite, comprising:
a biodegradable polyester-based polymer particle;
a magnetic nanoparticle dispersed in the biodegradable polyester-based polymer particle; and
a drug loaded in the biodegradable polyester-based polymer particle.

2. The magnetic field-sensitive nanocomposite according to claim 1, wherein the biodegradable polyester-based polymer particle comprises at least one of poly(L-lactic acid) (PLA), polyglycolic acid (PGA) and poly(lactic-co-glycolic acid) (PLGA).

3. The magnetic field-sensitive nanocomposite according to claim 1 , wherein
the biodegradable polyester-based polymer particle has a glass transition temperature (Tg) of 50° C. or less.

4. The magnetic field-sensitive nanocomposite according to claim 1, wherein
the magnetic field-sensitive nanocomposite comprises 1 to 80 parts by weight of the magnetic nanoparticle with respect to 100 parts by weight of the polymer particle.

5. The magnetic field-sensitive nanocomposite according to claim 1, wherein
the magnetic nanoparticle has an average diameter of 1 to 50 nm.

6. The magnetic field-sensitive nanocomposite according to claim 1, wherein
the magnetic nanoparticle comprises a superparamagnetic iron oxide nanoparticle.

7. The magnetic field-sensitive nanocomposite according to claim 1, wherein
the drug comprises at least one of a diagnostic drug, a therapeutic drug, and a drug for a reaction reagent.

8. The magnetic ⁻field-sensitive nanocomposite according to claim 1, wherein
the biodegradable polyester-based polymer particle has an average diameter of 900 nm or less.

9. A method of preparing a magnetic field-sensitive nanocomposite, comprising:
(a) mixing an organic solvent, a magnetic nanoparticle and a biodegradable polyester-based polymer;
(b) adding distilled water in which a drug is dispersed to a resultant product of the step (a), and stiffing the resultant product with the distilled water to form a first emulsion;
(c) adding distilled water in which an emulsifier is dissolved to the first emulsion and stirring the first emulsion with the distilled water to form a second emulsion; and
(d) drying the second emulsion to prepare a magnetic field-sensitive nanocomposite.

10. The method of preparing a magnetic field-sensitive nanocomposite according to claim 9, wherein
in the step (a), 1 to 80 parts by weight of the magnetic nanoparticle are mixed with respect to 100 parts by weight of the biodegradable polyester-based polymer.

11. The method of preparing a magnetic field-sensitive nanocomposite according to claim 9, wherein
In each of the steps (b) and (c), the stirring is performed using ultrasonic waves.

12. The method of preparing a magnetic field-sensitive nanocomposite according to claim 9, wherein
the biodegradable polyester-based polymer comprises at least one of poly(L-lactic acid) (PLA), polyglycolic acid (PGA) and poly(lactic-co-glycolic acid) (PLGA).

13. The method of preparing a magnetic field-sensitive nanocomposite according to claim 9, wherein
the biodegradable polyester-based polymer has a glass transition temperature (Tg) of 50° C. a less.

14. The method of preparing a magnetic field-sensitive nanocomposite according to claim 9, wherein
the magnetic nanoparticle has an average diameter of 1 to 50 nm.

15. The method of preparing a magnetic field-sensitive nanocomposite according to claim 9, wherein
the magnetic nanoparticle comprises a superparamagnetic iron oxide nanoparticle.

16. The method of preparing a magnetic field-sensitive nanocomposite according to claim 9, wherein
the drug includes at least one of a diagnostic drug, a therapeutic drug, and a drug for a reaction reagent.

17. A system for anticancer treatment, comprising:
the magnetic field-sensitive nanocomposite according to claim 1,
wherein the magnetic nanoparticle generates heat when an alternating magnetic field is applied to the magnetic field-sensitive nanocomposite, and the generated heat causes the biodegradable polyester-based polymer particle to undergo a phase-transition or decomposition such that the drug is released.
